(19) United States
(12) Patent Application Publication
(10) Pub. No.: US 2006/0084076 A1
(43) Pub. Date: Apr. 20, 2006

(54) Method for the amplification of genetic information

(75) Inventor: Wolfgang Mann, Neudrossenfeld (DE)

Correspondence Address: HOUSTON ELISEEVA, 4 MILITIA DRIVE, SUITE 4, LEXINGTON, MA, 02421, US

(73) Assignee: Alopex GmbH, Kulmbach (DE)

(21) Appl. No.: 11/077,193

(22) Filed: Mar. 10, 2005

Related U.S. Application Data
(63) Continuation of application No. PCT/EP03/10132, filed on Sep. 11, 2003.

(30) Foreign Application Priority Data
Sep. 12, 2002 (DE) 102 42 359.8

(51) Int. Cl. C12Q 1/68; C12P 19/34

(52) U.S. Cl. 435/6; 435/91.2

(57) ABSTRACT

The invention relates to a method for the amplification of genetic information, wherein it is possible to analyze the frequency of unlimited partial amounts of genetic material from very small sample amounts. The inventive methods enable parallel amplification of specific target sequences of the respective partial amounts and simultaneous qualitative analysis thereof and subsequent relative quantitative analysis, wherein several high concentration amplification products containing many target sequences and qualitatively high value amplification products are provided.

### RELATED APPLICATIONS

This application is a Continuation of PCT application serial number PCT/EP2003/010132 filed on Sep. 11, 2003 (which was published in German under PCT Article 21(2) as International Publication No. WO 2004/027089 A1) which claims priority to German Application No. DE 102 42 359.8, filed on Sep. 12, 2002, both of which are incorporated herein by reference in their entirety.

### BACKGROUND OF THE INVENTION

In the case of humans, childless couples or women may be offered assisted reproduction to fulfill their wishes for children. Currently there are several procedures available for this purpose, leading to an average pregnancy rate of around 10% per egg cell. Indirect analyses conducted via the polar bodies of the egg cell have shown that a high percentage of egg cells have a maldistribution for individual chromosomes. These aneuploidies lead to non-viable embryos and presumably account for the low implantation rate after assisted reproduction.

In the population around 10% of all couples are unintentionally childless. The reasons for infertility lie on the one hand in organic defects in the woman or the man, but these may also have genetic causes. For up to 70% of miscarriages genetic reasons, mainly chromosomal maldistribution, may be held responsible (Griffin 1996).

These chromosomal aneuploidies are mostly due to a faulty oogenesis (Angell 1993). In the spermiogenesis, only 2-4% aneuploidal cells are found (Zenses 1992). These and other causes lead to the fact that, under natural conditions, a high percentage of all fertilized egg cells do not lead to an intact pregnancy.

As already mentioned above, childless couples or women may be offered in vitro fertilization or another assisted reproduction method, such as e.g. intracytoplasmic sperm injection (ICSI). With the in vitro fertilization methods currently available, an average pregnancy rate of 10% per egg cell can be obtained. The analysis of large studies has revealed that the pregnancy rate in women over 35 years of age declines markedly, and is below 10% for women over 40. This goes along with the observation that mothers over 35 years of age carry an increased risk of a child with chromosomal maldistribution.

In prenatal diagnosis, only those children with a chromosomal maldistribution are diagnosed who are viable at least up to the time of diagnosis. Thus e.g. at the time of amniocentesis, more children are found with trisomia or monosomia than at the time of birth, since many of these children die in the course of pregnancy.

If one considers just the trisomia, then essentially only children with a trisomia 21 or with one surplus or missing X or Y chromosome are viable. The low implantation rates referred to above may be accounted for by aneuploidies of the egg cells for other chromosomes too, leading either to no implantation or to a very early abortion. This is supported by chromosome analyses on aborted material.

During egg cell maturation, the initially diploid egg cell must reduce its chromosome complement. This process is completed in the first and second maturation division. In the first maturation division (1st reduction division), the homologous chromosomes are separated. In the second maturation division, the chromatids are separated. The genetic material of the resultant daughter cells is transferred in each case in the form of polar bodies into the perivitelline space of the egg cell. In their structure, polar bodies correspond to a cell, but have only minimal cytoplasm content. The first polar body occurs during ovulation, while the second polar body is extracted 3-4 h after the sperm has penetrated the egg cell. The two polar bodies differ in the amount of their genetic material. The first polar body contains 23 chromosomes with 2 chromatids (2n), while the second—like the mature egg cell—has only 23 simple chromosomes with only one chromatid (1n). The polar bodies have no function whatsoever and are resorbed in the early development of the embryo. There is no known biological significance of the polar body for the embryo (see abstract of “Preimplantation Genetic Diagnosis, Polar Body Biopsy” from the First World Congress on: Controversies in Obstetrics, Gynaecology & Infertility, Prague, Czech Republic-1999 by Y. Verlinsky, A. Kuliew, and flyer on Polar Body Diagnosis from the Prenatal Medical Centre, Munich, Dr. med. Karl-Philip Gloning et al. The analysis of first and second polar bodies by means of “sequential testing”, which assumes sampling of the polar body, is disclosed in the abstract of “Preimplantation Genetic Diagnosis, Polar Body Biopsy” from the First World Congress on: Controversies in Obstetrics, Gynaecology & Infertility, Prague, Czech Republic-1999 by Y. Verlinsky, A. Kuliew. 179 successful artificial pregnancies resulted in 135 healthy children, who suffered no damage from this intervention.

M. Montag, K. van der Ven, H. van der Ven “Erste klinische Erfahrungen mit der Polkörperchendiagnostik in Deutschland”)[“First Clinical Experiences with Polar Body Diagnosis in Germany”] report on first experiences with polar body diagnosis in Germany, according to which pregnancy rates using polar body diagnosis are gratifyingly high.

“Einführung in die Präimplantationsdiagnostik”, [“Introduction to Pre-implantation Diagnosis”] E. Schwinger, Lübeck, Source: http://www.studgen.uni-mainz.de/manuskripte/schwinger.pdf states that no increase in the malformation rate after PID by polar body or blastocyst analysis can be found. The document makes clear the narrow time windows available for pre-fertilization diagnosis (PFD).

According to a flyer on Polar Body Diagnosis from the Prenatal Medical Centre, Munich, Dr. med. Karl-Philip Gloning et al., the results published to date suggest that polar body sampling is not associated with any appreciable increase in the general basic risk of 2-4% for developmental anomalies.

There are thus already human beings in existence who have come from egg cells from which the first polar body was removed, and who have suffered no damage from this intervention. Polar body analysis therefore suggests itself as the method of choice for testing egg cells for their suitability for successful fertilization.

The polar bodies represent the number of chromosomes in the egg cell and are available for the conduct of a genetic analysis.

Individual cell analysis on a polar body assumes its sterile removal from the perivitelline space of the egg cell. The classical removal method is to open the zona pellucida using a micro-manipulator, with subsequent isolation of the polar body.

The possibility of precise observation of the fertilization process during in vitro fertilization (IVF) has revealed that some of the egg cells can not be fertilized, or that already fertilized egg cells do not divide further. Many of these frustrated fertilization attempts are probably due to aneuploidies of the egg cells. Several working groups have been occupied with the genetic analysis of polar bodies. For these studies the polar bodies were isolated and subjected to a fluorescence in situ hybridization (FISH). In this analysis, molecules specific to certain chromosomes and marked with a fluorescent dye are hybridized on the chromosomes of the polar bodies. If then a deviant number of signals are found for a chromosome, an aberrant chromosome distribution during oogenesis is indicated. With the aid of this method, used for the analysis of 3943 oocytes in 1999, it was found that 43% of oocytes had a chromosomal maldistribution, with the latter occurring in both the first and the second maturation division. In this study only chromosomes 13, 18 and 21 were analyzed in respect of their correct distribution (Verlinsky 1998). A refinement of the technique now permits the simultaneous analysis of 5 different chromosomes. The method is basically limited in the number of chromosomes which may be analyzed, since each chromosome requires the use of a different fluorochrome and an unambiguous evaluation is possible only when the signals do not overlap.

Known from U.S. Pat. No. 6,143,564 is a method for variation of the genetic information of the egg cells of animals with the aid of polar bodies.

Known from JP 2086800 is a method for proving the existence of a specific gene in a fertilized egg cell, in which the first and the second polar bodies are analyzed.

Chromosome-specific catcher molecules for in-situ hybridizations by the FISH method are known from U.S. Pat. No. 5,817,462. Here, through various combinations of different fluorophores, all human chromosomes may be detected simultaneously. With greater numbers of chromosomes, the number of required combinations of suitable fluorophores becomes increasingly complex, likewise the analysis. If necessary, individual chromosomes must then be differentiated under the microscope with the aid of their size.

FISH experiments are therefore suitable only to a limited extent for simultaneous quantification of the chromosomes within a genetic material. The quantification of delimitable partial amounts of a genetic material by such methods is limited for the time being to chromosomes. The number of dyes which may be combined is limited, and in the case of a polar body analysis, the polar body is consumed after a FISH analysis has taken place. To date it has proved impossible to make a reliable statement concerning the integrity of a complete chromosome complement by the aforementioned method. This is also due to the fact that the preparation for FISH hybridizations involving polar bodies can not be carried out as in the case of the established FISH procedure on metaphase cells, since the genomic DNA of a polar body may not be divided further, and since the chromosomes of a polar body may not be condensed like conventional chromosomes.

Methods of chromosome banding or conventional homogeneous dyeing besides the FISH technique have not proved to be satisfactory, since widely ranging anomaly rates have been found depending on the method used in various studies (Eckel et al. 2001).

Known from U.S. Pat. No. 6,060,251 is a method for determining the chromosomal identity of a sample containing genomic DNA, in which the genomic DNA is amplified and provided with marking agents. The amplification method described is an unspecific amplification method in which repetitive sequences are used as primer binding sites. The amplification product is then analyzed using a DNA library. Detection is made through the detection of hybrids, wherein the catcher molecules from the DNA library may be applied to a solid carrier.

In principle it is possible to make a statement concerning the existence of a chromosome in a sample by amplifying, as part of a specific amplification, a target sequence which occurs only on a specific chromosome, and then detecting this target sequence by creating a catcher-target sequence hybrid. To enable such catcher-target sequence hybrids to be detected, a minimum quantity of them is required, and a corresponding minimum amount of copies of the target sequence must be generated, for which reason amplification is necessary. Since in the course of a specific amplification, as a rule only copies of a specific target sequence are generated, detection of the relevant hybrids only allows a conclusion regarding the existence of chromosomes which have this target sequence. Known from WO 00/24925 are methods and means for determining the chromosomal composition of a cell, in which the genetic material to be analyzed is first of all amplified by means of an unspecific PCR amplification, in which polar bodies are likewise named as the source of such genetic material. The following PCR methods are cited: DOP-PCR, primer extension amplification PCR, ligation mediated PCR, tagged PCR and alu-PCR. In these amplification methods, extremely unspecific primers ensure that a representative chromosome complement of the genetic material present in a cell is amplified. Besides target sequences which may be assigned to individual chromosomes, a multiplicity of completely unspecific sequence occur therein. The amplification product may be analyzed using a genetic chip. With the methods described it should be possible to detect chromosomal differences and aneuploidies. At the same time, in addition to the sample to be analyzed, a parallel reference sample is amplified. The two samples are provided with different marking agents and applied to a chip which has catcher molecules able to form hybrids with the target sequences concerned. However, due to the multiplicity of co-amplified unspecific sequences, the following problems arise:
the target sequences which it is ultimately important to detect occur diluted in a mixture with a multiplicity of completely unspecific sequences
the multiplicity of co-amplified unspecific sequences may include sequences which are so similar to the target sequences that the catcher molecules form hybrids which in detection are interpreted to the effect that the target sequence assigned to the respective catcher molecule is present, which does not then correspond to reality or only to a limited extent
the dilution of target sequences within a multiplicity of unspecific sequences, which is due directly to the non-specific nature of the amplification method, requires a higher number of cycles to produce a minimum quantity of target sequences which can lead to a detectable minimum quantity of catcher-target sequence hybrids; however, each cycle increases the risk of amplification products with defects, leading in turn to difficulties in the creation of the desired “correct” catcher-target sequence hybrid and its distinction from undesired faulty hybrids
with high numbers of cycles, the creation of PCR products based on a target sequence no longer increases exponentially but instead stagnates, from a certain cycle onwards. The cycle from which this occurs depends amongst other things on the initial concentration of this target sequence. Consequently, with simultaneous amplification by PCR and different initial concentrations of the target sequences, the increases in concentration of different amplified target sequences of a genetic material begin to stagnate at different stages of amplification. It is then no longer possible to make a statement on the relative quantity of these target sequences.

According to WO 00/24925, the product obtained from the unspecific amplification may undergo a subsequent specific amplification, so that a statement may be made on the existence of the target sequence of the specific amplification in the original material. With a specific amplification, only a quite specific target sequence is amplified. The product of the second amplification therefore permits a statement only concerning the existence of the target sequence amplified in that case. Comparison of the relative quantity of products of different second amplification experiments leads, owing to imponderables in the preceding unspecific amplification, to no useful statement. There is also the fact that each amplification experiment in itself is influenced by a multitude of parameters which are difficult to reproduce. Inevitably, therefore, the results of these combined amplification processes are subject to fluctuation. A simultaneous analysis of such target sequences under conditions of maximum comparability is therefore not possible using the methods known from WO 00/24925. Moreover, a concrete statement regarding the existence of a target sequence of a cell analyzed in accordance with WO 00/24925 requires a laborious procedure which is costly in material and in time.

Described in the PubMed database of NCBI, address www.ncbi.nlm.nih.gov., abstract on the rapid detection of common autosomal aneuploidies by quantitative fluorescent PCR on uncultured amniocytes, RAHIL. H. et al, Eur. J Hum. Genet. (August 2002) 10(8) 462-6, is a co-amplification of DSCR1, DCC and RB1 in which a separate primer pair is required for each of these genes, consequently three primer pairs for the three gene regions.

Database PubMed of NCBI, address www.ncbi.nlm.nih.gov., abstract on: Identification of chromosomal translocations in leukaemia by hybridization with oligonucleotide microarrays, NASEDKINA, T. et al., Haematologica (April 2002) 87 (4) 363-72 and Database PubMed of NCBI, address www.ncbi.nlm.nih.gov., abstract on: DNA microarray technology for neonatal screening, DOBROWOLSKI, S. F. et al, Acta Paediatr. Suppl. (1999) 88 (432) 61-4 describe multiplex PCR reactions, with several different sequences being amplified simultaneously.

WO 02/44411 describes a method of detecting aneuploidies based on expression profiling. This involves identifying the expression of genes which occur on a chromosome, and from this determining the chromosome.

Methods conducted with the aid of chromosome spreading are known from WO 00/24925.

EP 1 026 260 A1 describes the analysis of tissue samples and mRNA, consequently of material from a multiplicity of cells.

DE 101 02 687 A1 and DE 100 59 776 A1 are concerned with the detection of aneuploidies, but the methods described are not usable for detecting aneuploidies starting with a single cell or even a polar body.

U.S. Pat. No. 6,329,140 outlines principles and possible uses of DNA chip technology in conjunction with methods for the selection of cloned organisms.

Known from EP 1 026 260 is a method for the simultaneous determination of gene expression and genetic abnormalities using DNA arrays, in which the DNA array described is suitable for gene expression and for the detection of chromosomal abnormalities in a tissue sample. For this purpose, the chip is provided with catcher molecules which may be assigned to specific chromosomes. Expressed and non-expressed sample material may be distinguished from one another using this method and the DNA chip described.

### SUMMARY OF THE INVENTION

In general it may be said that expansion of the technical aids for the conduct of analyses concerning the existence of delimitable partial amounts and their relative quantity within a genetic material, in particular for chromosome analyses on polar bodies, is welcome.

Under the Embryo Protection Law of 13.12.1990, the conduct in Germany of pre-implantation diagnosis for the detection of chromosomal anomalies in human embryos is forbidden and their selection is not possible. This rules out analysis of the second polar body of human egg cells. When the first polar body develops, however, the egg cell is not yet fertilized and, so long as no fertilization has taken place, the egg cell is not the subject of the Embryo Protection Law. Consequently an improvement in pregnancy rates could be obtained through the cytogenetic analysis of the first polar body. This would involve detection of aneuploidal oocytes before fertilization, and these could then be excluded from the further fertilization process (Eckel et al. 2001). For this, only a limited period of time is available within which the egg cell may be fertilized with success. This period of time varies between 1-2 days.

A method of this kind could also prove useful in the reproduction of other vertebrates, e.g. in the reproduction of species threatened with extinction. In such cases, too, analysis of the second polar body would not in principle be forbidden. On sampling of the second polar body, however, the time available before implantation of the fertilized cell is generally distinctly less than the time available after sampling of the first polar body.

Since egg cells may be fertilized successfully only within a short period of time, the method should be quick and should allow the most reliable statement possible concerning the relative quantity of the individual chromosomes.

The problem of the invention is therefore to provide a method for the amplification of genetic material which makes possible the simultaneous quantitative analysis of delimitable partial amounts within a genetic material, available in a very small quantity, and which is suitable for detection of the chromosomes present in a polar body and their quantity relative to one another.

The problem is solved by a method with the features of the independent claims. Advantageous developments thereof are specified in the further dependent claims.

The present invention relates to methods for the amplification of genetic information of a genetic material, in which the genetic information may be assigned to delimitable partial amounts of the genetic material.

Partial amounts within genetic material are for example chromosomes within a genome. In this case a chromosome represents a partial amount of a genome containing several different chromosomes. Delimitable partial amounts may however also be deletions and/or insertions within an individual chromosome. In this case the deletions and/or insertions represent the delimitable partial amounts and the individual chromosome the genetic material. Genetic information from a chromosome is e.g. target sequences which can only occur on this chromosome, i.e. are specific for this chromosome.

A system in which the delimitable partial amounts represent different chromosomes of a genome is e.g. a polar body.

Polar bodies develop in vertebrates during the formation and maturation of egg cells, which are needed to reproduce the type concerned.

The problem is solved by a method for the amplification of genetic information from genetic material containing several partial amounts delimitable from one another, by means of polymerase chain reaction in which primers are used which are complementary to primer binding sites present in the genetic material at several points, and which are adjacent to a target sequence of predetermined length and specific to one partial amount in each case. Thus an amplification product is obtained which substantially has only amplified sequences containing a target sequence of predetermined length and specific for the genetic information concerned, which is suitable for detection by hybridization.

The amplification method according to the invention is used to amplify simultaneously target sequences which are different, and are each specific for one partial amount of the genetic material. These target sequences are all amplified under the same reaction conditions. They are present in the product of the amplification method according to the invention in a significantly higher concentration than in unspecific amplification methods according to the prior art. As a result, fewer cycles are required than under the prior art in order to produce quantities of target sequences detectable by hybridization. This is accompanied by a reduced rate of error in amplification, and with a lower number of faulty hybridizations in the event of detection. The product of the method according to the invention thus permits more rapid, more reliable and more meaningful analyses than is possible with products of known amplification methods in which many different sequences are amplified at the same time.

The amplification product created by the method according to the invention contains substantially only amplified sequences containing a target sequence of predetermined length, specific for the genetic information concerned, and suitable for detection by means of hybridization. Substantially, at least 80%, preferably 90% or 95% may involve specific target sequences.

For successful quantitative detection it is expedient for statistical reasons that the primer binding sites of the primers used are arranged adjacent to at least 10, 20, 30, 50 or 100 specific target sequences of a delimitable partial amount, so that at least 10, 20, 30, 50 or 100 specific target sequences are amplified per delimitable partial amount.

The problem is also solved by a method with the following steps:
conduct of a method for the amplification of genetic information from genetic material containing several partial amounts of genetic material delimitable from one another, so that an amplification product is obtained with sequences containing target sequences which may be assigned to the delimitable partial amounts, and which is suitable for detection by hybridization
mingling the amplification product with catcher molecules on a DNA chip, so that hybrids of catcher molecules and partial amounts of the amplification product are formed, wherein the DNA chip contains at least two groups of spots, wherein the spots within a group have different catcher molecules, and each group of spots may be assigned to one of the delimitable partial amounts of the genetic material
quantitative detection of the hybrids formed in each case in a spot with different catcher molecules of the DNA chip, so that for each spot a detection value is obtained,
averaging of the detection values of the groups of spots present on the DNA chip
determination of the relative frequency of partial amounts of genetic material within a genetic material by comparison of the averages.

A method of this kind according to the invention, using a DNA chip, permits an averaging of the detection values of signals which may be assigned to delimitable partial amounts of the genetic material, and has the advantage that the method has a high degree of tolerance against the reinforcement of otherwise disadvantageous effects associated with the amplification process.

In a method according to the invention in which the homology between the primers and the respective primer binding sites lies in a range of 80-100%, and preferably in a range of 90-100%, the content of target sequences in the amplification product is especially high.

In a method according to the invention in which the distance between primer binding sites and the adjacent specific target sequences is no more than 1000, preferably no more than 300, and in particular no more than 100 bases, the time required to implement the method is low and the proportion of target sequences in the amplification product is especially high.

In a method according to the invention in which the predetermined lengths of the specific target sequences are between 15 and 80 bases, and preferably between 20 and 50 bases, the specificity of the target sequences for the respective portions may be ensured very easily.

In a method according to the invention in which all specific target sequences are of substantially the same length, a pool of target sequences is obtained which form hybrids with catcher oligonucleotides provided for their detection and which have very similar properties. E.g. the hybrids have similar melting temperatures when they are of equal length, i.e. they are similarly stable, and e.g. the formation of such hybrids proceeds at comparable speeds.

In a method according to the invention in which, in the course of the polymerase chain reaction, nucleotide components provided with markings are used, an amplification product is obtained which, after hybridization of the target sequences with corresponding catcher oligonucleotides, is easily detected with the aid of the respectively incorporated marking.

In a method according to the invention in which the following steps are also taken:
mingling of the amplification product with catcher molecules, so that hybrids of catcher molecules and target sequences are formed, and
detection of the hybrids

the nature of the detected hybrids and the amount in which they are present may be used to make a statement concerning the amount and the existence of the respective delimitable partial amount in the genetic material.

In a method according to the invention in which the following steps are also taken:
mingling of the amplification product with catcher molecules, so that hybrids of catcher molecules and target sequences are formed, and
detection of the hybrids

and in which the catcher molecules are arranged on a DNA chip, all hybrids formed may be detected simultaneously and compared with one another in a very small space.

In a method according to the invention in which the following steps are also taken:
mingling of the amplification product with catcher molecules, so that hybrids of catcher molecules and target sequences are formed, and
detection of the hybrids

and in which the catcher molecules are formed by oligonucleotides, a high degree of accuracy of hybridization may be ensured.

In a method according to the invention in which a DNA chip is used, in which in an individual spot in each case identical catcher molecules are provided, it is possible using the intensity of detection within this spot to make a statement regarding the existence of a specific target sequence within the amplification product.

In a method according to the invention in which a DNA chip is used, in which in an individual spot different catcher molecules for different target sequences are provided, all assigned to one of the delimitable partial amounts of the genetic material, the measurement of the intensity in such a spot is sufficient:
for a statement to be made regarding the existence of this delimitable partial amount in the genetic material, and
in comparison with the intensity of the other spots, for a reliable statement to be made regarding the relative quantity of the delimitable partial amount in the genetic material.

A method according to the invention in which the genetic material stems from or is traceable back to a single cell permits a quick, reliable and high quality statement on the quantity of delimitable partial amounts within the genetic material of the egg cell.

A method according to the invention in which the genetic material is a chromosome complement from a polar body of an egg cell permits a quick, reliable and high quality statement concerning the suitability of the egg cell for fertilization without the egg cell itself being damaged.

A method according to the invention in which a delimitable partial amount consists of one or more chromosomes permits a statement on the integrity of the chromosomal composition of a genetic material.

A method according to the invention in which a delimitable partial amount consists of one or more genes permits a statement on the existence of deletions or insertions within a genetic material.

A method according to the invention in which a genetic reference material is amplified in parallel under otherwise identical reaction conditions, provides an amplification product which on the one hand permits the determination of the delimitable partial amounts of a genetic material and their quantity relative to one another, while also allowing a verification of this quantification to be made.

In contrast to a method as in D1, the method according to the invention has primers which are complementary to the primer binding sites which occur at several points within a genetic material, and which are each adjacent to a target sequence specific for each partial amount. This means that identical or substantially identical primer binding sites are adjacent to different target sequences, which in turn implies that an individual primer or an individual primer pair is in a position to amplify several different specific target sequences.

In comparison with multiplex PCR reactions, a significant feature of the method according to the invention is that here an amplification product is obtained which has substantially only amplified sequences containing a target sequence of predetermined length and specific for the genetic information concerned, and which are suitable for detection by means of hybridization.

Classical co-amplification of different primers or conventional PCR experiments start with large amounts of material, for example total DNA, cell cultures or the blood of new-born infants. Expression profiling methods start from mRNA, which represents a small selection of what is contained in genomic DNA. It involves a quite different starting material from for example the chromosomal DNA of a polar body. The polar body is not transcription-active and therefore contains no mRNA. All methods based on the quantification of mRNA are unsuitable for polar body analysis. Known methods are therefore unsuitable in particular for the detection of chromosome anomalies in a single cell. The method according to the invention is however able to perform this task.

For that reason, certainly all nucleic acids are suitable for detection by means of hybridization. Here however it is assumed that an adequate amount of material is available for the process of detection itself.

In any event, a single molecule or just a few molecules of a target nucleic acid, bound to a catcher, is or are insufficient for this purpose. Consequently the sample material, when the amount is below the detection threshold, must be amplified.

The method according to the invention permits a quantitative analysis based on the genomic DNA contained in a single cell. Such an analysis also facilitates other methods.

The method according to the invention is not limited to use only in connection with the material of a single cell. This represents only one application of the method according to the invention. The method is in fact suitable in principle for use in all cases involving the detection of partial amounts of a genetic material and their relative frequency within a total genetic material. There may be other methods for such detection. Of the known methods, however, none has the features and the advantages of the method according to the invention and is applicable to a single cell.

The problem as stated above, and the features and advantages of the present invention, may be better understood by taking into consideration the following detailed description of the figures, preferred variants, and an embodiment of the present invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a flow chart showing a selection procedure for the selection of target sequences.

FIG. 2 is a flow chart showing a selection procedure for the selection of primer binding sites and primers.

FIG. 3 shows in schematic form the luminous intensity of a selection of measuring points on the surface of a microarray according to the invention.

FIG. 4 is a plan view of an electrophoresis gel according to an embodiment of the invention;

FIG. 5 is a plan view of a band in an electrophoresis gel; and

FIGS. 6a, 6b are schematic representations of the surfaces of microarrays according to the invention.

In respect of features of the invention not explained in detail above, reference is made expressly to the patent claims and the figures.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### DETAILED DESCRIPTION OF THE PREFERRED EMBODIMENTS

The invention is explained in detail below with the aid of a first variant and the figures.

In the context of the first variant, the genetic material involves the genomic DNA (sperm) present in a haploid chromosome complement, and the delimitable partial amounts involve all chromosomes Chr which may occur in the chromosome complement. The chromosome complement is a human sperm, for which reason the number of delimitable partial amounts is 23. This corresponds to the number of possible chromosomes Chr 1-Chr 23 present in the chromosome complement.

For each delimitable partial amount Chr 1, Chr 2, . . . Chr 23 there are target sequences which are only part of one or of a limited number of the delimitable partial amounts. This means that these target sequences are unique for the chromosome or chromosomes concerned, on which these target sequences occur. For each chromosome there is a multiplicity of such specific target sequences.

For the method according to the invention, suitable primers are determined in a selection process in two stages.

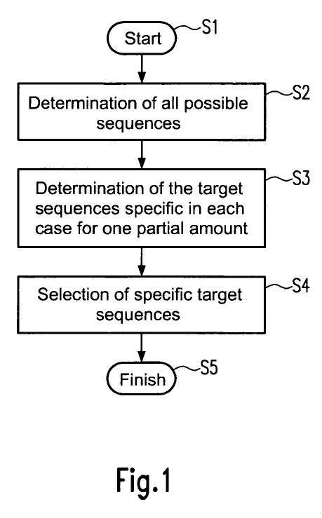

FIG. 1 shows in schematic form a selection process for the selection of target sequences. This method begins with step S1. In step S2, all possible target sequences for all delimitable partial amounts are determined. This means that all possible amplifiable sections in the genetic material are determined. It is then determined (S3) which of these target sequences are specific for a single delimitable partial amount in each case. Delimitable partial amounts may be e.g. chromosomes. A target sequence is specific when it occurs in only one single delimitable partial amount but not in several delimitable partial amounts.

For each delimitable partial amount, several different target sequences are selected (S4). Preferably the target sequences are selected on the basis of certain criteria, e.g. target sequences which are highly distinctive are preferred to other target sequences. In other words, target sequences with the lowest possible homology or complementarity to other target sequences are preferred in selection. It is also useful to select target sequences with similar hybridization properties (e.g. melting temperature, formation rate).

The selection process ends with step S5.

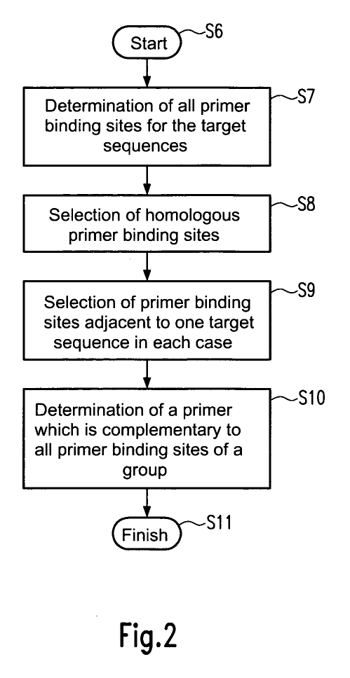

In a second process section (FIG. 2), in each case a primer is determined which is suitable for the amplification according to the invention, and specifically in a selection process with the following steps:

a) within the genetic material, primer binding sites are determined (S7) which are located in the vicinity of the 3′-end of the target sequences determined in the first step. In an amplification reaction, a primer hybridized at these primer binding sites is extended beyond the target sequence and a complement to the target sequence is produced.

b) from the primer binding sites determined in a), a selection is made (S8) of those which are substantially homologous to one another. Here, those primer binding sites with a low homology to other primer binding sites determined under a) are rejected. Essentially homology means that the primer binding sites have a homology of at least 80% to one another.

c) from the primer binding sites determined in a), a selection is made (S9) of those which are substantially in the vicinity of the 3′-end of a target sequence or its complement. In this context, proximity essentially means only that at least 50% of the primer binding sites are in the vicinity of the 3′-end of a target sequence or its complement. These primer binding sites are combined to form a group of primer binding sites. For each of the primer binding sites of this group a primer is determined (S10) which is substantially complementary to all primer binding sites of the group. In this context, complementary means essentially that the primer, under suitable reaction conditions, will form hybrids with all primer binding sites of the group.

The primer binding sites of the group do not necessarily include the primer binding sites required for amplification of all target sequences. For this purpose, a primer binding site in the vicinity of the respective 3′-end of the target sequence is required at both strands of a target sequence, so that the latter is flanked in each case by two primer binding sites.

If this is the case, then the second process step may be repeated, so as to determine one or more further groups of primer binding sites and the associated primers.

In this way, one or several primers are selected which form hybrids with all primer binding sites required for amplification of the target sequences. That is to say, the primer binding sites are substantially to be found only in the vicinity of the 3′-end of the target sequences or their complements.

The amplification only of target molecules means essentially, in the context of the invention, that at least 50% of the amplified molecules are target molecules containing target sequences which are specific for at least one delimitable partial amount, but can not be traced back to all delimitable partial amounts.

Naturally the selection process described may be subject to various iteration processes, i.e. various of the specified criteria may be given different weightings and individual steps may be interchanged or repeated several times depending on previously obtained results. In particular, this may also mean that unspecific primers known in a first step may be used, allowing the amplification of the target sequences described above and only afterwards being checked for conformity with the criteria (specificity of the target sequences, distinctiveness, similar hybridization properties, etc.) of the first step.

Within the scope of the selection process described, unspecific primers according to the prior art, such as used e.g. in the context of DOP-PCR or inter-ALU-PCR, may be so modified that they conform to the selection criteria cited above.

The available genetic material of the chromosome complement undergoes an amplification process according to the invention. In this, the primer or primers in each hybridization bind to primer binding sites located in the vicinity of the 3′-end of target sequences, so that substantially only target molecules containing the target sequences are amplified.

In the amplification product, each chromosome is represented by a number of different target sequences specific for the chromosome concerned and which is specific for this chromosome.

The amplification reaction follows the formula Y=Sx(1+E)n, wherein Y is the number of copies of an amplified target sequence produced, E is the efficiency of amplification, n the number of cycles, and S the number of originally existing “start copies” of a particular target sequence (a target sequence specific for a chromosome may occur several times on the chromosome concerned).

In the sperm or in a polar body of a normally developed egg cell, the chromosomes occur in each case only once. In the event of chromosome maldistribution, certain chromosomes are present in a different number, e.g. 0 or 2.

This means that, in the amplification of target sequences with only a single molecule as start copy (S=1) it must be ensured experimentally for a quantitative statement, that in the first cycle of amplification a defined chromosome-specific target sequence is detected and amplified with certainty. In respect of an individual molecule, however, this is not generally possible. If the first cycle fails, then at the end only half the copies of these target sequences will be amplified. The error in amplification may lie in a greater range in which it is also intended to quantify (factor 1, 2, 3 . . . ) the frequency with which a chromosome is represented in a sperm or polar body. Quantitative statements with a single molecule as start sequence are therefore subject to such great uncertainty as to be in fact worthless.

The same applies to efficiency E, which amounts to 1 only in the ideal case, i.e. in each cycle of the amplification a doubling of the starting material, i.e. all available copies, takes place. In reality, though, ideal efficiency never occurs, and the value for E must always be set <1. Efficiency is incidentally dependent on a multitude of factors which are difficult to control, e.g. on the sequence amplified in the particular case, and on the length of the amplified sections of a genome. It varies in principle from one experiment to another. Small deviations in efficiency E from the ideal efficiency of an amplification 1 lead to very great effects in typical cycle numbers for amplification processes of n=20-30.

With the aid of the method according to the invention, for each chromosome present in the sperm or polar body, a multiplicity of different target sequences is amplified, virtually all (at least 80%) of them specific for at least one chromosome, and specifically with the aid of one or more primers. Experimental imponderables, due to the fluctuating efficiency of the amplification process from one experiment to another, are ruled out by the fact that all target sequences are amplified simultaneously in a single process. Errors in amplification, resulting from the failure to amplify certain target sequences of a chromosome in the first cycle, are offset by the fact that in any event a substantial portion of the target sequences which are specific for a chromosome are amplified in the first step.

If e.g. the first chromosome Chr1 of a chromosome complement contains 26 target sequences a-z, which occur only on this chromosome and are amplified simultaneously with the aid of a method according to the invention using one or more primers, and if the target sequences a, b are not amplified in the first cycle of the amplification, but the target sequences c-z are amplified in the first step, then the error relating to target sequences a, b is not significant in the amplification product, so long as ultimately the totality of the amplified target sequences a-z specific to the chromosome is used to provide a statement concerning the quantity of the chromosome in the sperm or polar body.

The amplification product may then be applied to a DNA chip on the surface of which are spots arranged in rows and columns, each with identical catcher molecules. The catcher molecules may form catcher-target sequence hybrids with the target sequences concerned. Here a suitable spot on the chip is provided for each target sequence or for the overwhelming majority of the target sequences. Depending on the probe molecules located on them, the spots are specific for one target sequence and therefore specific for at least one chromosome.

When the amplification product is applied to such a DNA chip under hybridization conditions, catcher-target sequence hybrids are formed, and these are then detected. If the amplification has been made using nucleotide triphosphates provided with fluorescent markers, it is possible to measure the fluorescence intensity of the individual spots.

Those spots Chr1a-Chr1z which are to be assigned to the target sequences a-z of the chromosome Chr1, are
if the chromosome was never present in the chromosome complement, those with no fluorescence or only a very small amount which is due to impurities
if the chromosome was present once or more in the chromosome complement, those with an average fluorescence intensity IChr1.

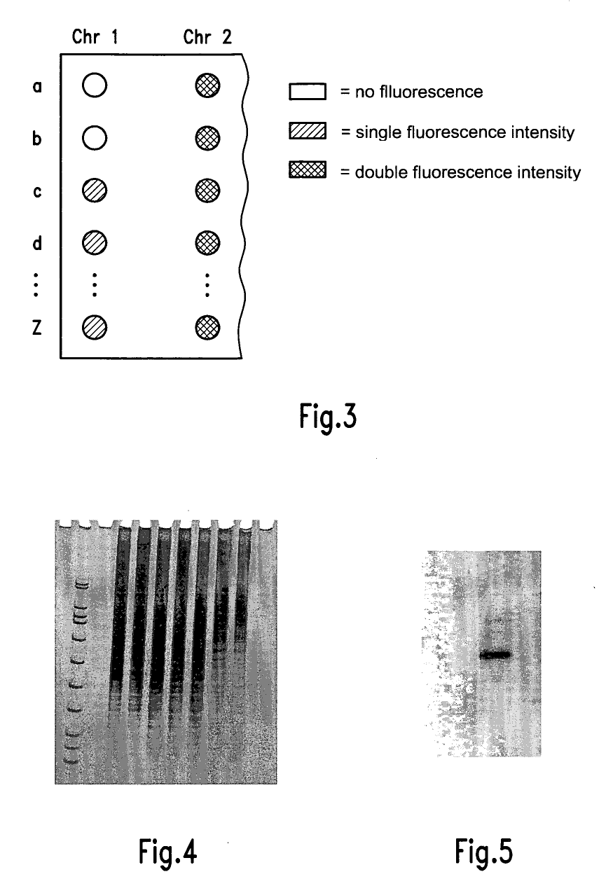

If target sequences a, b of chromosome 1 are amplified with poor efficiency, this leads to spots Chr1a, Chr1b in which no or only minimal fluorescence intensity is measured, shown in FIG. 3 as measuring points without hatching. If the other target sequences c-z are amplified with high efficiency, then a correspondingly high fluorescence intensity is measured in the spots Chr1c-Chr1z, shown in FIG. 3 as measuring points with line hatching. If the chromosome Chr1 was present in the chromosome complement once, and chromosome 2 was present in the chromosome complement twice, then the average intensity IChr1 of the fluorescence of the spots Chr1a-Chr1z assigned to chromosome 1 will be half that of the average intensity IChr2 of the fluorescence of the spots Chr2a-Chr2z assigned to chromosome 2, shown in FIG. 3 by cross-hatching.

It may occur that a target sequence aa, which is specific for chromosome Chr 1, is at the same time specific for a further chromosome, but not for all chromosomes of a chromosome complement. If both chromosomes occur frequently in a sample, the intensity of the fluorescence measured in the spot assigned to this target sequence aa will be approximately twice that measured in spots, the target sequence of which occurs only on one chromosome.

For the analysis of the product of the amplification according to the invention, a multiplicity of further hybridization experiments is available to the person skilled in the art. Thus the amplified sequences may for example also be analyzed by means of electrophoresis methods, capillary electrophoresis or mass spectrometry.

The invention will be explained in detail below with the aid of a second variant.

The genomic information of a human chromosome complement is amplified by means of an amplification method, in which the amplification product contains a multiplicity of target sequences, and in which each chromosome present in the polar body may be assigned target sequences which occur only on this chromosome or may stem from it.

For this purpose the amplification method according to the above embodiment may be implemented, but unspecific amplification methods according to the prior art may also be used, while in principle other methods may also be used as PCR methods, e.g. using NASBA, Qβ replicase, or SDA (see K. Hagen-Mann, W. Mann, 1995, Exp. Clin. Endocrinol 103: 150-155). Here it is important only that all or as many as possible of the target sequences are contained in the amplification product, i.e. that the target sequences are amplified in parallel.

The amplification product is brought into combination with a DNA chip on which each chromosome is represented by 10 spots. At the same time each spot contains catcher oligonucleotides which are able to form hybrids with target sequences, these hybrids being specific for one chromosome. One spot contains 10 different catcher oligonucleotides which are able to form hybrids with target sequences; these hybrids differ from one another but are all assigned to the same chromosome. The same applies to the other nine spots which are assigned to the same chromosome. In the case of a chromosome Chrn, of which 26 target sequences a-z may be captured on the chip by catcher oligonucleotide, the spots are mixed as follows: the first spot Chrn/1 contains catcher molecules for the target sequences a-j, the second spot Chrn/2 contains catcher oligonucleotides for the target sequences j-t, the third spot Chrn/3 contains catcher oligonucleotides for the target sequences u-d, the fourth spot Chrn/4 contains catcher oligonucleotides for the target sequences e-o, the fifth spot Chrn/5 contains catcher oligonucleotides for the target sequences p-z, the sixth spot Chrn/6 contains catcher oligonucleotides for the target sequences a, c, e, g, i, k, m, o, q, t, the seventh spot Chrn/7 contains catcher oligonucleotides for the target sequences b, d, f, h, j, 1, n, p, r, t, the eighth spot Chrn/8 contains catcher oligonucleotides for the target sequences m, n, o, p, q, r, w, y, z, v, the ninth spot Chrn/9 contains catcher oligonucleotides for the target sequences a, e, i, j, m, n, o, p, r, s and the tenth spot Chrn/10 contains catcher oligonucleotides for the target sequences a, b, c, d, e, v, w, x, y, z. For each of the 23 chromosomes Chr1-Chr23 of a chromosome complement which may be present in a chromosome complement of a human egg cell, the chip is provided with 10 such spots Chrn/1-Chrn10, on which in each case 10 of 26 catcher oligonucleotides are mixed as detailed above. These catcher oligonucleotides are able to hybridise with target sequences which have been basically amplified in the course of an unspecific amplification, if the chromosome for which the relevant target sequences are specific is present in the chromosome.

The amplification product is applied to the DNA chip. Here the catcher oligonucleotides hybridise with the target sequences a-z of each chromosome which are complementary to them. As part of the amplification, a marking agent is incorporated in the amplified target sequences (a Cy-3 fluorescent marker). The chip is washed, and the fluorescence of the individual spots is determined simultaneously. This involves detecting the intensity I_{Chrn/x} of each individual spot x assigned to a chromosome n. All intensities I_{Chrn/x} of a chromosome n are used in averaging the intensity of the spots which are specific for a chromosome (resulting mean intensity: Iₙ). The intensities I₁-I₂₃ are compared with one another. If the order of magnitude of the mean intensity of the spots assigned to a chromosome=approximately 0, then this chromosome is not contained in the chromosome complement. If the mean intensity of the spots assigned to a chromosome has a value corresponding to the majority of the other intensities, then from this it is concluded that the chromosome to which these spots are assigned occurs in the chromosome complement exactly once. If the mean intensity of the spots representing one chromosome is twice, three times or several times the other intensities, then it is assumed that these chromosomes occur in the chromosome complement twice, three or four times or more often.

The frequency of specific target sequences within a chromosome may be high or low. This frequency is where applicable to be taken into account by determining a suitable factor, and also the effect of the frequency of start copies of a target sequence on the formation of specific hybrids in a spot after carrying out a parallel amplification. The frequency of the target sequences of a specific chromosome may also depend on the size of the chromosome concerned. Resultant effects are if applicable also to be incorporated in a suitable correction factor, which is used in the analysis.

It is very unlikely that all chromosomes of a chromosome complement occur in it twice, for which reason the statement made with the aid of the method according to the invention, regarding the quantity of chromosomes in a chromosome complement, is very reliable. To enhance this reliability, however, a reference sample may be amplified in parallel, and analyzed simultaneously with the sample for analysis.

If one of the spots of such a DNA chip is faulty for production reasons, e.g. because it was poorly spotted, then nine further spots are still available to allow statements to be made on the relative quantity of a chromosome in the chromosome complement. Through the mixing in one spot of catcher sequences which are different for one chromosome, but specific for different target sequences from this chromosome, each spot will have a measurable intensity—even with unequal efficiency of amplification with regard to the target sequence concerned—so long as suitable starting material was present in the chromosome complement, corresponding to a statistical mean. Each spot in itself is therefore more meaningful than a spot in which only one type of catcher molecule has been provided. Through the presence of several such mixed spots per chromosome, which also contain different mixed catcher molecules, inaccuracies in amplification are more readily excluded than in previous methods.

If the measured intensities of the first, second, third . . . tenth mixed spots 1, 2, 3 . . . 10 which are each assigned to one of the chromosomes Chr1-Chr23 of a chromosome complement are set in relation to one another, then 10 different statements are obtained on the quantitative occurrence of the up to 23 chromosomes normally occurring in a human chromosome complement. This equates to a multiple verification of the analysis result.

Instead of 10 different spots as just described, it is also possible to provide just one spot for each chromosome which—according to a variant of the embodiment—contains 26 catcher molecules corresponding to the target sequences a-z of a chromosome. Arithmetical averaging is unnecessary—the mean intensity of all hybrids specific to a chromosome is obtained through the mixing of the catcher molecules in one spot.

From the number determined for the chromosomes present in a chromosome complement, a direct conclusion may be made as to the number of chromosomes in the egg cell. In this way the chromosomal integrity of an egg cell may be determined with a high level of confidence.

Catcher molecules in the context of the invention preferably comprise synthetic oligonucleotides. However they may also contain: DNA, cDNA, RNA, aRNA, LNA and/or other modified nucleic acids.

The invention is described below with the aid of a specific embodiment. For the amplification of the chromosome material, in each case isolated from a single cell, the following primer was selected in accordance with the method of FIG. 2:

| | | |
|---|---|---|
| Ale1-k | 5′-CCAAAGTGCTGGGATTACAG-3′ | (SEQ ID NO.: 1) |

With this primer sequence, a PCR amplification is conducted under the following conditions: several different samples are first of all heated for 5 minutes to 95° C., then for 35 times 30 seconds to 95° C., 30 seconds to 62° C. and 30 seconds to 72° C. At the end of the last cycle, the samples are heated for 10 minutes to 72° C. and then cooled down to 4° C.

The primer Ale1-k has proved to be extremely efficient in the conduct of the method according to the invention. In the replacement of only one base by another base the primer is still able to carry out its function, in particular when only the terminal primer sections are affected. Even with the omission of two terminal bases from the primer, useful results can still be obtained. Such variations, which are known to the person skilled in the art, do however lead to considerable loss of quality. If more than two bases of the primer according to the invention are replaced or omitted, then the method according to the invention is scarcely capable of implementation. The fact that the primer fulfils its function is explained below with the aid of FIGS. 4 and 5.

The amplification products are applied to a gel and subjected to a gel electrophoresis. A view of the resultant electrophoresis gel is shown in FIG. 4. On this, arranged from left to right, 9 traces 1-9 may be recognized. Trace 1 is the molecular weight standard, trace 2 a negative control, and traces 3-9 are identical specimens of different samples, each with one haploid cell as starting material.

Detectable on the gel shown in FIG. 4 are sequences which have been predicted in silico in accordance with a method as shown in FIG. 1.

By way of example, two sequences are specified: SHGC-6833 and RH102636, which are to be found under their respective designations in the NCBI database. Both sequences are part of the specific sequences amplified by means of Ale1-k. SHGC-6833 is to be found specifically on chromosome 21. The sequence, SEQ ID NO.:2, (hereafter described as sequence tagged site sequence or STS sequence) of SHGC-6833 reads:

SEQ ID NO.: 2

acagaaaggtggaggaaaagttagagcaatattttttggtttatagctgg
ctttggggaaaacggattctggtttctatgcctagcctcagggaaacgtg
agatggataacatgagggcaggagaaggtcagacgaaaacttttgcttcc
aaggtctttgttttgagtatcattttctgaatcccgacattccctg
gtctgaaactttcccaagaagtttcacagtccagaaattggattggt

By hybridizing with a marked STS probe, i.e. a complement to the STS sequence, in which a marking agent is incorporated, on to the gel shown in FIG. 4 (trace 3), a specific signal of the anticipated size is obtained, as shown in FIG. 5. This signal is the proof of the existence of SHGC-6833 in the starting material and thus for the existence of chromosome 21.

RH102636 is to be found specifically on chromosome 1. The sequence of RH102636, SEQ ID NO.: 3, reads as follows:

SEQ ID NO.: 3

ccatgtaacacaagctcacagcctctaatgttaccaaccttataca
caaatggccaaacaagaaattgtcctttccaaaagataatttattc
tggtttcccctcttca

The detection of RH 102636 on the gel is at the same time proof that RH102636 was present in the starting material and thus the proof for the existence of chromosome 1.

The sequence concerned occurs only a single time on the particular chromosome. At the point where the fluorescence intensity of the two sequence traces is roughly the same, it may be stated that chromosomes 1 and 21 are present in equal amounts in the sample concerned.

In silico, further sequences have been predicted, each occurring only a single time on a particular chromosome. Table 1 gives a summary of the sequences predicted to date.

TABLE 1
Summary of all sequences predicted in silico

| Chromo- | | of which STS- | Number of primer binding |
|---|---|---|---|
| some | PCR-Product | sequences | sites |
| Chr. 1 | 4512 | 30 | 71485 |
| Chr. 2 | 3016 | 8 | 59619 |
| Chr. 3 | 2245 | 10 | 45920 |
| Chr. 4 | 1664 | 11 | 38816 |
| Chr. 5 | 2076 | 16 | 40794 |
| Chr. 6 | 2124 | 6 | 41695 |
| Chr. 7 | 3350 | 31 | 49076 |
| Chr. 8 | 1608 | 9 | 34000 |
| Chr. 9 | 1966 | 8 | 33818 |
| Chr. 10 | 2268 | 9 | 39827 |
| Chr. 11 | 1894 | 11 | 34259 |
| Chr. 12 | 2429 | 17 | 40262 |
| Chr. 13 | 1195 | 7 | 26921 |
| Chr. 14 | 2162 | 22 | 34280 |
| Chr. 15 | 2198 | 12 | 35623 |
| Chr. 16 | 3677 | 8 | 46664 |
| Chr. 17 | 4255 | 23 | 52336 |
| Chr. 18 | 984 | 4 | 21969 |
| Chr. 19 | 6049 | 24 | 54283 |
| Chr. 20 | 1958 | 10 | 27451 |
| Chr. 21 | 571 | 15 | 11533 |
| Chr. 22 | 1872 | 9 | 22584 |
| Chr. X | 1834 | 8 | 32452 |
| Chr. Y | 160 | 1 | 4585 |

The first column in the table lists the respective human chromosome. The second column gives the number of different amplification products of an amplification with Ale1-k predicted in silico for the chromosomes concerned. Almost all of the amplification products in the second column are specific. Given in the third column is the number of formerly known and published specific sequences (STS sequences) for the particular chromosome, which are accessible in public databases and represent in each case a partial amount of the relevant PCR products in column 2. The fourth column shows the number of primer binding sites for Ale1-k on the chromosome concerned. Since the primer does not always have a binding site in the required proximity to a first binding site for successful amplification of a section, and at which it may also hybridise a complement in the reverse direction, a PCR product does not always automatically result. With the primer Ale1-k according to the invention, this occurs in only a fraction of cases. Accordingly it might be assumed that with 71485 primer binding sites of chromosome 1 around 35000 PCR products would be obtained, but their number is only 4512.

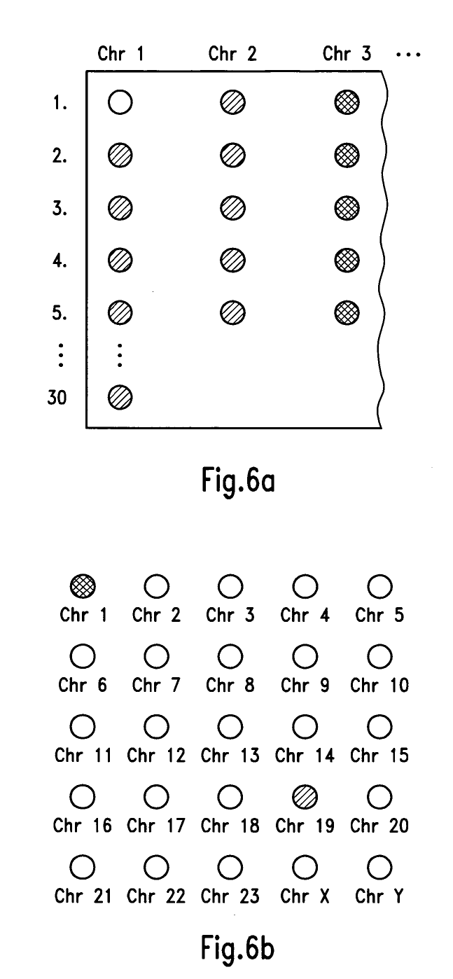

A DNA chip or microarray used for analysis of a reaction mixture obtained from an amplification according to the invention may be designed as shown in schematic form in FIG. 6a or FIG. 6b.

One option is to provide only one separate measuring point for each STS sequence. Such a measuring point contains only catcher molecules which will form a hybrid specifically with the relevant STS sequence or a section thereof. A fluorescence trace at a measuring point then indicates that this sequence was present in the sample. For chromosome 1, for example, up to 30 different measuring points may be provided on a microarray. If in the course of an amplification, one of 30 of the STS sequences detectable on the microarray for chromosome 1 is poorly amplified, for example because in the first amplification cycle in this sequence the primer did not bind to the primer binding site provided, there are still 29 further sequences available, the detection of which is at the same time proof of the existence of chromosome 1 in the sample. If an amplification error of this kind leads to a lowering of the amplified amount of this sequence relative to the other sequences then, in the measuring point representing this sequence, a lower fluorescence intensity will be observed than in the other measuring points (measuring point without hatching, top left in FIG. 6a). Due to the fact that, for each of 29 other amplification products specific for chromosome 1, a measuring point is provided on the microarray, the faulty amplification product can be identified as such. Only some of the other 29 measuring points (line-hatched measuring points 2-29 in the first column of the microarray shown in FIG. 6a) are then used in the analysis to determine the relative amount of chromosome 1. If their intensity is roughly equal to the intensity measured for measuring points specifically representing in each case one STS sequence of chromosome 2, then it follows that the amounts of the respectively amplified STS sequences of chromosomes 1 and 2 are approximately equal (column 2 of the measuring points in FIG. 6a with line hatching). From this it follows in turn that chromosomes 1 and 2 are present in the sample concerned in the same relative proportions. If chromosome 3 occurs in the sample twice as often as the other chromosomes, then the corresponding measuring points will show twice the fluorescence intensity of the other measuring points (measuring points with cross-hatching, third column in FIG. 6a). Individual measuring points which, due to amplification errors, which occur regularly in the course of amplification from a very small amount of starting material, have no or only minimal fluorescence intensity, as just described with reference to the first measuring point for chromosome 1, provide no impediment to the analysis so long as at least one STS sequence per chromosome is correctly amplified. The probability that, due to amplification errors, all STS sequences of a chromosome have been more poorly amplified than the STS sequences of another chromosome, is statistically very low. A microarray on which catcher molecules are provided at different measuring points and are present there in equal concentrations, and which each form hybrids with a specific STS sequence from Table 1, is therefore ideally suited to provide, in a rapid and reliable manner, a statement regarding the relative amount of the chromosomes in a sample which has been amplified by Ale1-k.

One measuring point of a microarray according to the invention may also contain catcher molecules which are able to hybridise with all STS sequences which are specific for a certain chromosome, or with a certain number of such sequences. Such a microarray is shown schematically in FIG. 6a. At each spot, the chromosome it is intended to detect is shown. If at an individual measuring point, all STS sequences are detectable which are each specific for one of the chromosomes of Table 1, then the intensities of the detected hybrids relative to one another behave in the signal analysis like the number of STS sequences detected for each chromosome (i.e. maximum around 1:10, chromosome 19: chromosome 1, shown in FIG. 6b by cross-hatching in spot Chr1 and line hatching in spot Chr19; the remaining spots or measuring points are not hatched for reasons of clarity).

Also suitable is a microarray in which, at individual measuring points, different but not all STS sequences which are specific for a chromosome are detectable. These are to be weighted accordingly in the analysis of the measured intensities. Finally, different types of measuring point may be integrated on one microarray, i.e. the microarray may have measuring points conforming to those in FIG. 6a, measuring points conforming to those in FIG. 6b, or measuring points as just described. The integration of a multitude of different types of measuring point on a single microarray makes available all of the possible types of analysis described, so that the results may be more easily verified. This makes the method according to the invention especially reliable.

In the variants cited and in the embodiment, the invention has been explained with the aid of a sperm analysis. Methods according to the invention may also be applied to other genetic material besides the genome contained in a sperm, in particular to the genome and its chromosomes contained in a single human cell or in a human polar body. The method according to the invention may also be applied to specific deletions or insertions as delimitable partial amounts within a genetic material, e.g. within an individual chromosome or a section thereof as genetic material.

### RELEVANT PUBLICATIONS

Angell, R. R., Man, J. & Keith, J. 1993: Chromosome anomalies in human oocytes in relation to age. Hum Reprod. 8(7): 1047 54.
Beier, M. & Hoheisel, J. (1999): Nucl. Acids Res. 27: 1970 1977.
Eckel, H., Kleinstein, J., Wieacker, P., Stumm, M (2001): Die zytogenetische Analyse von nicht fertilisierten Oozyten—Möglichkeiten und Grenzen. [The cytogenetic analysis of non-fertilised oocytes—options and limits] medizinische Genetik 13: 25-30.
Garvin, A M, Holzgreve, W. & Hahn, S. (1998): Highly accurate analysis of heterozygous laci by single cell PCR. Nucl. Acids Res. 26: 3468 3472.
Griffin, D. K. (1996): The incidence, origin, and etiology of aneuploidy. Int Rev Cytol. 167: 263-96.
Grothues, D., Cantor, C. R. & Smith, C. (1993): PCR amplification of megabase DNA with tagged random primers (T PCR). Nucl. Acids Res. 21: 1321 1322.
Hagen-Mann, K. & Mann, W. (1990): Polymerase Chain Reaction-Eine revolutionäre Methode für die Biologie. [A revolutionary method for biology] BIUZ 20: 257 262.
Hagen-Mann, K. & Mann, W. (1995): RT-PCR and alternative methods to PCR for in vitro amplification of nucleic acids. Exp. Clin. Endocrinol. 103: 150-155.
Hardt, T., H. Himmelbauer, W. Mann, H. M. Ropers & Haaf, T. (1999): Towards identification of individual homologous chromosomes: comparative genomic hybridization and spectral karyotyping discriminating between paternal and maternal euchromatin in Mus musculus×M. spretus interspecific hybrids. Cytogen. Cell Genet. 86:187 193.
Heller, A., Chudoba, L, Bleck, C., SENGER; G., Claussen, U., & Liehr, T. (2000): CGH of Microdissection based comparative genomic hybridization analysis micro
   secondary acute myiegenous leukaemias. Int. J. Oncol. 16: 461 468.
Huang, Q, Schantz, S. P., Rao, P. H., Mo, J., McCormick, S. A. & Chaganti, R. S. (2000): Improving degenerate oligonucleotide primed PCR comparative genomic hybridization for analysis of DNA copy number changes in tumors. Genes Chromosomes Cancer 28: 395 403.
Kingsley, K, Wirth, J., van der Maarel, S., Freier, S., Ropers, R R & Haaf, T. (1997): Complex FISH probes for the subtelomeric regions of all human chromosomes: comparative genomic hybridization of CEPH YACs to chromosomes of the old world monkey Presbytis cristata and great apes. Cytogenet Cell Genet 78: 12 19.
Klein, C. A., Schmidt-Kitler, 0., Schardt, J. A., Pantel, K, Speicher, M. R. & Riethmuller, G. (1999): Comparative genomic hybridization, loss of heterozygosity, and DNA sequence analysis of single cells. PNAS 96: 4494 4499.
Pollack, J R, Perou, C. M., Alizadeh, A. A., Eisen, M R, Pergamenschikov, A., Williams, C. F., Jeffrey, S. S., Botstein, D. & P. O. Brown (1999): Genome wide analysis of DNA copy number changes using cDNA microarrays. Nature Genetics 23: 41 46.
Schnell, S. & Mendoza, C. (1997): Theoretical description of the polymerase chain reaction. J. theor. Biol. 188: 313 318.
Stolovitzky G. & Cecchi G. (1996): Efficiency of DNA replication in the polymerase chain reaction. PNAS 93:12947 12952.
Swaminathan, N., McMaster, K, Skowron, P. M., & Mead, D. (1998): Thermal cycle labeling: Zeptomole detection sensitivity and microgram probe amplification using CvU1 restriction generated oligonucleotides. Anal. Biochem. 255: 133 141.
Telenius, H. Carter, N. P., Bebb, C. E., Nordenskj61d, M., Ponder, B. A. J., & Tunnacliff, A. (1992): Degenerate oligonucleotide primed PCR: general amplification of target DNA by a single degenerated primer. Genomics 13: 718 725.
Verlinsky, Y., Cieslak, J., Ivakhnenko, V., Evsikov, S., Wolf, G., White, M., Lifchez, k Kaplan, B., Moise, J., Valle, 1, Ginsberg, N., Strom, C. & Kuliev, A. (1998): Preimplantation diagnosis of common aneuploidies by the first and second polar body FISH analysis. J Assist Reprod Genet.: 15(5):285 9.
Voullaire, L., Wilton, L., Slater, H. & Williamson, R. (1999): Detection of aneuploidy in single cells using comparative genomic hybridization. Prenat. Diagn. 19: 846 851.
Wodicka, L., Dong, H, Mittmann, M., Ho, M. H., Lockhart, D. 1(1997) Genome wide expression monitoring in Saccharpmyces cerevisiae. Nature Biotechnol 15: 1359-1367.
Zenzes, M. T., Wang, P. & Casper, R. F. (1992): Evidence for maternal predisposition to chromosome aneuploidy in multiple oocytes of some in vitro fertilization patients. Fertil Steril. 57(1):143 9.
Zhang, L., Cui, X., Schmitt, K, Hubert, R., Navidi, W. & Arnheim, N. (1992): Whole genome amplification from a single cell: implications for genetic analysis. PNAS 89:4847 5851.
Zhou, Y, Wang, H., Wie, J., Cui, L. Deng, X., Wang, X. & Chen, Z. (2000): Comparison of two PCR techniques used in amplification of microdissected plant chromosomes from rice and wheat. Biotechniques 28: 766 774.
Database PubMed at NCBI, address www.ncbi.nlm.nih.gov., Abstract: Rapid detection of common autosomal aneuploidies by quantitative fluorescent PCR on uncultured amniocytes. RAHIL, H. et al., Eur. J. Hum. Genet. (August 2002) 10 (8) 462-6.
Database PubMed at NCBI, address www.ncbi.nlm.nih.gov., Abstract: Identification of chromosomal translocations in leukaemias by hybridization with oligonucleotide microarrays. NASEDKINA, T. et al., Haematologica (April 2002) 87 (4) 363-72.
Database PubMed at NCBI, address www.ncbi.nlm.nih.gov., Abstract: DNA microarray technology for neonatal screening. DOBROWOLSKI, S. F. et al., Acta Paediatr. Suppl. (1999) 88 (432) 61-4.
WO 02/44411
WO 00/24925
EP 1 026 260A1
DE 101 02 687 A1
DE 100 59 776 A1
Abstract on “Preimplantation Genetic Diagnosis, Polar Body Biopsy” from The First World Congress On: Controversies in Obstetrics, Gynaecology & Infertility, Prague, Czech Republic—1999 by Y. Verlinsky, A. Kuliew.
Journal für Fertilität und Reproduktion, [Journal for Fertility and Reproduction] Number 4/2002, page 7ff, M. Montag, K. van der Ven, H. van der Ven “Erste klinische Erfahrungen mit der Polkörperchendiagnostik in Deutschland” [Initial clinical experience with polar body diagnosis in Germany].
“Einführung in die Präimplantationsdiagnostik”, [“Introduction to Pre-implantation Diagnosis”] E. Schwinger, Lübeck, Source: http://www.studgen.unimainz.de/manuskripte/schwinger.pdf.
Flyer zu Polkörperchendiagnostik der Pränatal-Medizin München, [flyer on Polar Body Diagnosis from the Prenatal Medical Centre, Munich] Dr. med Karl-Philip Gloning et al.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for determining the relative frequency of delimitable partial amounts of genetic material, comprising:
a. amplifying the genetic material by means of polymerase chain reaction using primers which are complementary to primer binding sites present in the genetic material at several points, and which are adjacent to one or more target sequences of predetermined length or lengths and specific to one partial amount in each case, so that an amplification product is obtained which substantially has only amplified sequences containing the one or more target sequences; and
b. detecting the amplification product to determine the relative frequency of the delimitable partial amounts of genetic material.

2. The method of claim 1, wherein more than one target sequences are amplified and wherein the more than one target sequences are the same or different.

3. The method of claim 1, wherein homology between the primers and the respective primer binding sites is in a range of 80-100%, and preferably in a range of 90-100%.

4. The method of claim 1, wherein a distance between primer binding sites and adjacent specific target sequences is no more than 1000, preferably no more than 300, and in particular no more than 100 bases.

5. The method of claim 1, wherein the predetermined length or lengths is between 15 and 80 bases, and preferably between 20 and 50 bases.

6. The method of claim 1, wherein more than one target sequences are amplified and wherein the more than one target sequences have about the same length.

7. The method of claim 1, wherein the amplification product is analyzed by a hybridization experiment for determining the relative frequency of delimitable partial amounts.

8. The method of claim 1, wherein the polymerase chain reaction includes using nucleotide components provided with markings.

9. The method of claim 1, further comprising:
i. contacting the amplification product with catcher molecules to form hybrids of catcher molecules and target sequences; and
ii. detecting the hybrids.

10. The method of claim 8, wherein the catcher molecules are arranged on a DNA chip.

11. The method of claim 8, wherein the catcher molecules are formed by oligonucleotides.

12. The method of claim 1, wherein the method includes using a DNA chip in which identical catcher molecules are provided in an individual spot.

13. The method of claim 1, wherein identical catcher molecules are provided in an individual spot on a DNA chip.

14. The method of claim 1, wherein the method includes using a DNA chip in which different catcher molecules for different target sequences, all assigned to one of the delimitable partial amounts of the genetic material, are provided in an individual spot.

15. The method of claim 1, wherein different catcher molecules, for different target sequences, all assigned to one of the determinable partial amounts of the genetic material, are provided in an individual spot on a DNA chip.

16. A method for determining the relative frequency of delimitable partial amounts of genetic material according to claim 1, wherein the method comprises:
amplifying the genetic material containing several partial amounts of genetic material delimitable from one another, to obtain an amplification product suitable for detection by hybridization, the amplification product containing target sequences which may be assigned to the delimitable partial amounts;
contacting the amplification product with catcher molecules on a DNA chip, so that hybrids of catcher molecules and partial amounts of the amplification product are formed, wherein the DNA chip contains at least two groups of spots, wherein the spots within a group have different catcher molecules, and each group of spots may be assigned to one of the delimitable partial amounts of the genetic material;
quantitatively detecting the hybrids formed in each case in a spot with different catcher molecules on the DNA chip, so that for each spot a detection value is obtained;
averaging of the detection values of the groups of spots present on the DNA chip; and
determining the relative frequency of partial amounts of genetic material within a genetic material by comparison of the averages.

17. The method of claim 1, wherein the genetic material stems from or is traceable back to a single cell.

18. The method of claim 1, wherein the genetic material is a chromosome complement from a polar body of an egg cell.

19. The method of claim 1, wherein the delimitable partial amount consists of one or more chromosomes.

20. The method of claim 1, wherein the delimitable partial amount consists of one or more genes.

21. The method of claim 1, wherein genetic information of a genetic reference material is amplified in parallel, under otherwise identical reaction conditions, so that an amplification product is obtained which substantially has only amplified sequences containing a target sequence of predetermined length and specific for the genetic information concerned, which is suitable for detection by hybridization.

22. The method of claim 1, wherein at least 80%, preferably 90% or 95% of the primer binding sites are located adjacent to specific target sequences.

23. The method of claim 1, wherein the primer binding sites of the primer or primers used are arranged adjacent to at least 10, 20, 30, 50 or 100 specific target sequences of a delimitable partial amount, so that at least 10, 20, 30, 50 or 100 specific target sequences are amplified per delimitable partial amount.

24. The method of claim 1, wherein the primer used to conduct the amplifying step is primer Ale1-k, having the sequence of SEQ ID NO.: 1.

25. The method of claim 1, wherein the primer has the base sequence of

| | |
|---|---|
| 5′-CCAAAGTGCTGGGATTACAG-3′ | (SEQ ID NO.: 1) |

26. A method for determining the relative frequency of delimitable partial amounts of genetic material, comprising:
a. amplifying the genetic material containing several partial amounts of genetic material delimitable from one another, so that an amplification product is obtained with sequences containing target sequences which may be assigned to the delimitable partial amounts, and which is suitable for detection by hybridization;
b. contacting the amplification product with catcher molecules on a DNA chip, so that hybrids of catcher molecules and partial amounts of the amplification product are formed, wherein the DNA chip contains at least two groups of spots, wherein the spots within a group have different catcher molecules, and each group of spots may be assigned to one of the delimitable partial amounts of the genetic material;
c. quantitatively detecting the hybrids formed in each case in a spot with different catcher molecules of the DNA chip, so that a detection value is obtained for each spot;
d. averaging the detection values of the groups of spots present on the DNA chip; and
e. comparing the averages to determine the relative frequency of partial amounts of genetic material within the genetic material.

27. The method of claim 26, wherein the genetic material stems from or is traceable back to a single cell.

28. The method of claim 26, wherein the genetic material is a chromosome complement from a polar body of an egg cell.

29. The method of claim 26, wherein a delimitable partial amount consists of one or more chromosomes.

30. The method of claim 26, wherein a delimitable partial amount consists of one or more genes.

31. The method of claim 26, wherein genetic information of a genetic reference material is amplified in parallel under otherwise identical reaction conditions, so that an amplification product is obtained which substantially has only amplified sequences containing a target sequence of predetermined length and specific for the genetic information concerned, which is suitable for detection by hybridization.

32. The method of claim 26, wherein at least 80%, preferably 90% or 95% of the primer binding sites are located adjacent to specific target sequences.

33. The method of claim 26, wherein the primer binding sites of the primer or primers used are arranged adjacent to at least 10, 20, 30, 50 or 100 specific target sequences of a delimitable partial amount, so that at least 10, 20, 30, 50 or 100 specific target sequences are amplified per delimitable partial amount.

34. The method of claim 26, wherein the primer used to conduct the amplifying step is primer Ale1-k, having the sequence of SEQ ID NO.: 1.

35. The method of claim 26, wherein the primer has the base sequence 5′-CCAAAGTGCTGGGATTACAG-3′ (SEQ ID NO.: 1)

36. A primer for conducting an amplification, wherein the primer consists essentially of 5′-CCAAAGTGCTGGGATTACAG-3′ (SEQ ID NO.: 1).

37. A method of determining the relative frequency of delimitable partial amounts in genetic material, wherein the method comprises:
a. determining nucleic acid sequences of primers by a process that includes:
i. ascertaining the complementary primer binding sites present in the genetic material at more than one point, wherein the primer binding sites are adjacent to a target sequence of predetermined length and are specific to one partial amount;
ii. making the primer based on the ascertained complementary primer binding sites of step (i);
b) amplifying the genetic material by performing a polymerase chain reaction with said primers to obtain an amplification product having primarily only amplified sequences containing the target sequence of predetermined length and specific for the genetic material; and
d. detecting the amplification product to determine the relative frequency of delimitable partial amounts.

38. A method for determining the nucleic acid sequence of primers, wherein the method comprises the steps of:
a. ascertaining the complementary primer binding sites present in the genetic material at several points, wherein the primer binding sites are at least within about 1000 bases of a target sequence of a predetermined length of between about 15 and about 80 bases, and are specific to one partial amount;
b. obtaining the primer based on the ascertained complementary primer binding sites of step (a), wherein the identity between the primers and the respective primer binding sites is in a range between about 80% and about 100%.

39. A method for amplifying a nucleic acid sequence, wherein the method comprises the steps of:
a. ascertaining complementary primer binding sites present in a genetic material at several points, wherein the primer binding sites are adjacent to a target sequence of predetermined length and are specific to one partial amount;
b. obtaining the primer based on the ascertained complementary primer binding sites of step (a); and
c. amplifying the nucleic acid sequence by performing a polymerase chain reaction with said primers to obtain an amplification product having primarily only amplified sequences containing the target sequence of predetermined length and specific for the genetic material.

40. A method for determining the relative frequency of delimitable partial amounts of genetic material, comprising:
a. amplifying the genetic material by means of polymerase chain reaction using primers which are complementary to primer binding sites present in the genetic material at several points, and which are adjacent to a target sequence of predetermined length and specific to one partial amount in each case, so that an amplification product is obtained which substantially has only amplified sequences containing the target sequence, wherein one or more target sequences are amplified; and
b. detecting the amplification product to determine the relative frequency of the delimitable partial amounts of genetic material.


SEQUENCE LISTING (3 sequences)

SEQ ID NO: 1 - DNA, 20 bp, Artificial Sequence
  Primer, Ale1-k
ccaaagtgctgggattacag

SEQ ID NO: 2 - DNA, 243 bp, Homo sapien
  misc_feature (1)..(234): SHGC-6833
acagaaaggtggaggaaaagttagagcaatattttttggtttatagctggctttggggaaaacggattctggtttctatgcctagcctcagggaaacgtgagatggataacatgagggcaggagaaggtcagacgaaaacttttgcttccaaggtctttgttttgagtatcattttctgaatcccgacattccctggtctgaaactttcccaagaagtttcacagtccagaaattggattggt

SEQ ID NO: 3 - DNA, 108 bp, Homo sapien
  misc_feature (1)..(108): RH10263
ccatgtaacacaagctcacagcctctaatgttaccaaccttatacacaaatggccaaacaagaaattgtcctttccaaaagataatttattctggtttcccctcttca